(19) United States
(12) Patent Application Publication
(10) Pub. No.: US 2006/0167100 A1
(43) Pub. Date: Jul. 27, 2006

(54) Orodispersible pharmaceutical composition of an antithrombotic compound

(76) Inventors: Patrick Wuthrich, Orleans (FR); Herve Rolland, Olivet (FR); Marc Julien, Sigloy (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 10/564,137

(22) PCT Filed: Jul. 16, 2004
(86) PCT No.: PCT/FR04/01866

§ 371 Date: Jan. 10, 2006

(30) Foreign Application Priority Data
Jul. 18, 2003 (FR) 0308780

(51) Int. Cl. A61K 31/195; A61K 9/14

(52) U.S. Cl. 514/562; 424/489

(57) ABSTRACT

The invention relates to a solid orodispersible pharmaceutical composition of an antithrombotic compound (compound A), characterised in that it comprises compound A or a pharmaceutically acceptable salt thereof and granules consisting of co-dried lactose and starch.

The present invention relates to a solid orodispersible pharmaceutical form for the administration of an antithrombotic compound or a pharmaceutically acceptable salt thereof by the oral or buccal route.

The antithrombotic compound, hereinafter referred to as compound A, which is described in patent specification EP 648 741, is the compound of formula (I):

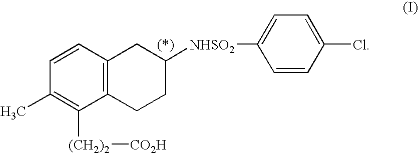

Compound A can be administered by the oral route in the form of tablets to be swallowed with half a glass of water.

The doses of compound A used by the oral or parenteral route to obtain the therapeutic effect generally range from 10 mg to 30 mg per administration, one or more times per day, in the form of an immediate-release tablet.

Many people have difficulty in swallowing conventional tablets, the size of which is often not negligible. The problems associated with the ingestion of medicines (choking; suffocation as a result of obstruction of the throat) are often the cause of poor compliance with dosage regimens or, indeed, of discontinuation of treatment.

The pharmaceutical compositions of the present invention make it possible not only to solve the known problems of a tablet form that has to be swallowed but also to offer a superior medical service which especially allows the quality of life of patients to be improved.

The orodispersible pharmaceutical composition of compound A has the advantage that elevated plasma levels of active ingredient are obtained rapidly and, moreover, by virtue of its rapid disintegration, makes it possible to limit the variations in absorption, which may be caused by various factors.

The orodispersible pharmaceutical composition according to the invention has the particular characteristic of requiring neither water nor chewing in the course of its administration. It disintegrates very rapidly in the mouth, preferably in less than three minutes and even more preferably in less than one minute.

Many rapid-dissolution forms are described in the prior art. In general, it is common to the previously described technologies that they use a disintegrating agent such as Kollidon® CL (crosslinked polyvinylpyrrolidone), EXPLOTAB® (carboxymethyl starch) and AC DISOL® (crosslinked sodium carboxymethylcellulose).

That disintegrating agent is indispensable to the formulation of the orodispersible tablets and has to be used in conjunction with a direct-compression excipient. The difficulties encountered in the manufacture of such tablets reside in the fact that it is very difficult to obtain tablets having physical characteristics that are constant and reproducible and compatible with the customary handling requirements of tablets.

However, the customarily used mixtures result in tablets of very considerable hardness which is completely unsuitable for rapid disintegration in the oral cavity.

Other orodispersible forms can be produced by using lyophilisation, resulting in very porous solid forms called “oral lyophilisates”. Those forms require the use of a highly specific and complicated industrial process which is lengthy to carry out, yielding a medicament form which has a high cost price.

The present invention enables those problems to be solved. It relates to a solid orodispersible form of compound A, optionally in the form of an optical isomer, or a pharmaceutically acceptable salt thereof, comprising a single excipient of natural origin which allows rapid disintegration and which has a neutral flavour and agreeable texture. The said excipient acts both as binder and as disintegrant. It allows a simple formulation of compound A to be obtained, having excellent suitability for direct compression, resulting in tablets of low friability and of a hardness that is compatible with customary handling methods.

More specifically, the invention relates to a solid orodispersible pharmaceutical composition of compound A or a pharmaceutically acceptable salt thereof, characterised in that it comprises:
compound A or a pharmaceutically acceptable salt thereof,
and granules consisting of co-dried lactose and starch.

Compound A preferably has the absolute configuration (R).

Preference is given to compound A being in the form of a sodium salt.

The composition according to the invention may also comprise, for reasons of manufacture, one or more lubricants and a flow agent and, for reasons of masking taste or bitterness, flavourings and sweetening agents as conventionally used.

In order to improve masking of the bitterness of compound A, the latter may optionally be associated with excipients such as cyclodextrins or coated with excipients using technologies known to the person skilled in the art such as, for example, coating in a fluidised-air bed, atomisation, coacervation, prilling and spray-congealing.

The invention relates also to the use of granules consisting of co-dried lactose and starch in the manufacture of solid orodispersible pharmaceutical compositions of compound A.

The term “orodispersible” is understood to refer to solid pharmaceutical compositions which disintegrate in the oral cavity in less than 3 minutes, preferably less than one minute.

The said granules present in the solid pharmaceutical compositions according to the invention correspond to the compositions described in Patent Application EP 00/402159.8. Those granules are characterised by a spherical structure and an advantageous compressibility and are marketed under the name STARLAC®.

The disintegrating properties of the said granules are known for tablets placed in large volumes of stirred liquids. It is especially surprising that, when used in the manufacture of orodispersible forms, the said granules should give especially satisfactory results in terms of disintegration in the mouth, for two reasons.

The first reason is based on the finding that the least water-soluble excipients are the most suitable for the formulation of orodispersible tablets (dissolution, in bringing about an increase in the viscosity of water, slows down its penetration into the tablets) and yet the said granules contain a large amount of highly water-soluble lactose. Moreover, the starch contained in the said granules is not a “super-disintegrant” agent as used and described in the orodispersible forms of the prior art.

The second is based on the finding that the disintegrant properties of an excipient (used in a tablet), when determined in water using conventional methods, cannot be extrapolated to the behaviour of the same tablet in vivo, in saliva. Disintegration rates in water are measured (in accordance with the European Pharmacopoeia) in an amount of water that is sufficiently large not to reach saturation level in terms of dissolution, whereas in vivo, by virtue of the small volume of saliva, the excipients are at saturation level. Furthermore, the stirring to which the tablets are subjected in the customary test does not reflect disintegration in the mouth. The Applicant accordingly found, during comparative tests, that certain excipients which are known as good disintegrants are not suitable for the preparation of orodispersible forms. Conversely, certain excipients that exhibit average disintegration in water may exhibit advantageous properties in vivo.

The Applicant then found, surprisingly, that the said granules rendered the tablets highly suitable for disintegration in the mouth, that being the case over a wide range of tablet hardness, whilst maintaining a low level of friability, which is especially remarkable. Most orodispersible forms of the prior art which disintegrate rapidly in the mouth are highly friable, which is reflected in the need to use a specific packaging and the risk of the tablet disintegrating as soon as it is handled and taken out of its pack.

It is especially remarkable that the above-mentioned criteria of orodispersibility and low friability are maintained over a wide range of tablet hardness, that is to say for tablets having a hardness of from 15 to 30 Newtons.

The pharmaceutical compositions according to the invention are preferably characterised in that they comprise, in relation to the total weight of the tablet:
from 2.5% to 20% by weight of compound A or a pharmaceutically acceptable salt thereof, preferably from 5% to 10%,
from 75% to 95% by weight of STARLAC®.

They may optionally comprise from 0.1% to 3% by weight of lubricating agents such as magnesium stearate, preferably from 0.5% to 1.5%, and from 0.1% to 3% by weight of a flow agent such as colloidal silica, preferably from 0.5% to 1.5%.

The following Examples illustrate the invention without limiting it in any way.

The orodispersible tablets were produced using the (R) isomer of compound A, in the form of a sodium salt.

### EXAMPLE 1

Formulation: Finished Tablet of 100 mg

| Constituents | Amount (mg) |
|---|---|
| Compound A, sodium salt | 10* |
| Starlac ® | 88.25 |
| Magnesium stearate | 1 |
| Anhydrous colloidal silica | 0.25 |
| Aspartame | 0.25 |
| Acesulfame K | 0.25 |

*expressed as compound A in the form of the base

### EXAMPLE 2

Formulation: Finished Tablet of 300 mg

| Constituents | Amount (mg) |
|---|---|
| Compound A, sodium salt | 30* |
| Starlac ® | 264.75 |
| Magnesium stearate | 3 |
| Anhydrous colloidal silica | 0.75 |
| Aspartame | 0.75 |
| Acesulfame K | 0.75 |

*expressed as compound A in the form of the base

The tablets are prepared by mixing the constituents, followed by direct compression. The hardness of the tablets of Examples 1 and 2 is about 15 Newtons and 30 Newtons, respectively.

In order to determine the disintegration time in the mouth, the orodispersible tablets of compound A described in Examples 1 and 2 were placed in the mouth. In these tests it was found that, for each of the formulations tested, the disintegration time in the mouth was less than 1 minute.

1-12. (canceled)

13- A solid orodispersible pharmaceutical composition comprising compound A of formula (I), optionally in the form of an optical isomer, or a pharmaceutically acceptable salt thereof: 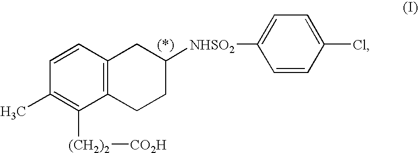
and granules consisting of co-dried lactose and starch.

14- The composition according to claim 13, wherein the composition disintegrates in the mouth in less than three minutes.

15- The composition according to claim 14, wherein the composition disintegrates in the mouth in less than one minute.

16- The composition according to claim 13, wherein compound A has the (R) configuration.

17- The composition according to claim 13, comprising, in relation to the total weight of the composition:
from 2.5% to 20% by weight of compound A or a pharmaceutically acceptable salt thereof, and
from 75% to 95% by weight of granules consisting of co-dried lactose and starch.

18- The composition according to claim 17, comprising from 5% to 10% by weight of compound A or a pharmaceutically acceptable salt thereof.

19- The composition according to claim 13, wherein compound A is in the form of a sodium salt.

20- The composition according to claim 13, further comprising one or more flavourings and sweeteners.

21- The composition according to claim 13, further comprising one or more lubricants and a flow agent.

22- The composition according to claim 13, wherein the composition is in the form of a tablet.

23- The tablet according to claim 22, wherein the tablet is obtained by direct compression.

24- The tablet according to claim 23, wherein the tablet has a hardness from 15 to 30 Newtons.

25- A process for the manufacture of solid orodispersible compositions of compound A, or a pharmaceutically acceptable salt thereof, which disintegrate in the mouth in less than three minutes, wherein compound A, or a pharmaceutically acceptable salt thereof, is mixed with granules consisting of co-dried lactose and starch.

26- A process for the manufacture of solid orodispersible compositions of compound A, or a pharmaceutically acceptable salt thereof, which disintegrate in the mouth in less than one minute, wherein compound A, or a pharmaceutically acceptable salt thereof, is mixed with granules consisting of co-dried lactose and starch.

27- A method for treating a living animal body, including a human, afflicted with a condition treatable by an antithrombotic agent, comprising the step of administering to the living animal body, including a human, a composition according to claim 13 which is effective for alleviation of the condition.
